(19) United States
(12) Patent Application Publication
(10) Pub. No.: US 2021/0003522 A1
(43) Pub. Date: Jan. 7, 2021

(54) ADIABATIC CONCRETE CALORIMETER AND METHOD

(71) Applicant: W. Calvin McCall, Charlotte, NC (US)

(72) Inventor: W. Calvin McCall, Charlotte, NC (US)

(21) Appl. No.: 16/502,575

(22) Filed: Jul. 3, 2019

(51) Int. Cl. G01N 25/20; G01N 33/38

(52) U.S. Cl. CPC G01N 25/20; G01N 33/383

(57) ABSTRACT

An adiabatic concrete calorimeter that has a thermal chamber which includes a cover and an insulated test cylinder container for containing a cylindrical concrete sample to be tested by being inserted and sealed into the thermal chamber. A plurality of temperature sensors is positioned in spaced-apart locations on the test cylinder container with leads connecting the sensors to a control box containing a data storage device.

### CROSS-REFERENCE TO RELATED PATENT APPLICATIONS

This patent application is a non-provisional application which traces priority to and claims the full benefit of U.S. Provisional Patent Application No. 62/692,989 filed on Jul. 2 2018, entitled “Adiabatic Concrete calorimeter and Method”, the contents of which are incorporated by reference herein.

### TECHNICAL FIELD AND BACKGROUND OF INVENTION

This application relates to an apparatus and method of directly measuring the quantity of heat and the rate of heat generation from a concrete specimen. As concrete elements gain strength due to a chemical process, the heat that is generated from this chemical process is typically referred to as the “heat of hydration.” For the purposes of this application “heat of hydration” can be defined as the heat that is generated from a specific concrete mixture over a specific period of time. An adiabatic concrete calorimeter is a device that directly measures the quantity of heat and the rate of heat generation from a concrete specimen. The device measures the quantity of heat generated by the concrete specimen by adding or subtracting heat from the calorimeter container. This evaluation method is different from a semi adiabatic calorimeter which measures the heat loss through the calorimeter container and mathematically calculates the temperature of the concrete specimen based on these measured losses.

In certain concrete applications the quantity of the “heat of hydration” needs to be measured. These temperature values are used to determine what, if any, precautions are needed to keep excessive temperatures and differential temperatures from developing between the interior and the surface temperature of the concrete element. These values must be evaluated and monitored so as they do not have a detrimental impact on the quality of the concrete element. The heat of hydration values that are measured by the adiabatic concrete calorimeter are used to develop concrete thermal control plans for specific concrete mixtures and specific concrete element sizes.

There is a need for an apparatus and method of measuring heat of hydration more accurately, efficiently and simply.

### SUMMARY OF THE INVENTION

It is therefore an object of the invention to provide an apparatus and method for determining the quantity of heat generated by a concrete specimen by adding or subtracting heat from a calorimeter container.

In accordance with one embodiment of the invention, a thermal chamber is provided that includes a cover for enclosing a test cylinder container containing a cylindrical concrete sample to be tested. Thermometers, for example, resistance temperature detectors (RTDs), are provided in spaced-apart locations on the test cylinder container with leads to a control box containing a data storage device, a suitable computing device and read outs. The test cylinder container includes standoffs for centering the test cylinder container in the thermal chamber. The thermal chamber is heavily insulated to reduce thermal loss through the side walls of the chamber.

These and other objects and advantages of the present invention are achieved in the preferred embodiments set forth below by providing an adiabatic concrete calorimeter having a thermal chamber which includes a cover and an insulated test cylinder container for containing a cylindrical concrete sample to be tested by being inserted and sealed into the thermal chamber. A plurality of temperature sensors is positioned in spaced-apart locations on the test cylinder container with leads connecting the sensors to a control box containing a data storage device.

### BRIEF DESCRIPTION OF THE DRAWINGS

FIG. 1 is a schematic side elevation of a thermal chamber and a test cylinder container adapted for being inserted into the thermal chamber and sealed against heat loss;

FIG. 2 is a schematic side elevation of the thermal chamber and test cylinder container of FIG. 1;

FIG. 3 is a vertical cross-section taken through lines A-A of FIG. 2 showing the test cylinder container correctly positioned in the thermal chamber; and

FIG. 4 is a horizontal cross-section taken through lines B-B of FIG. 2 showing the centered position of the test cylinder container in the thermal chamber.

### DETAILED DESCRIPTION OF PREFERRED EMBODIMENT

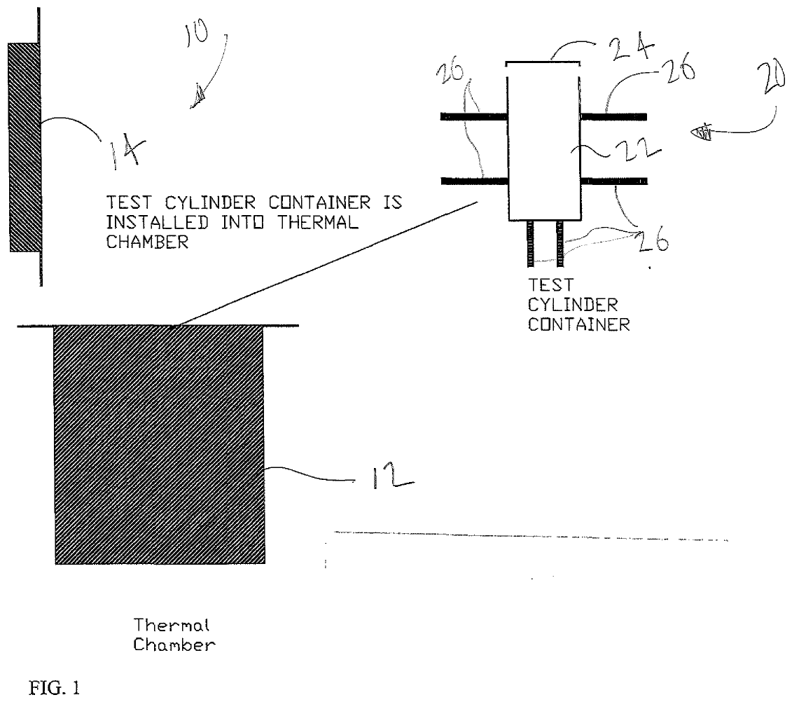

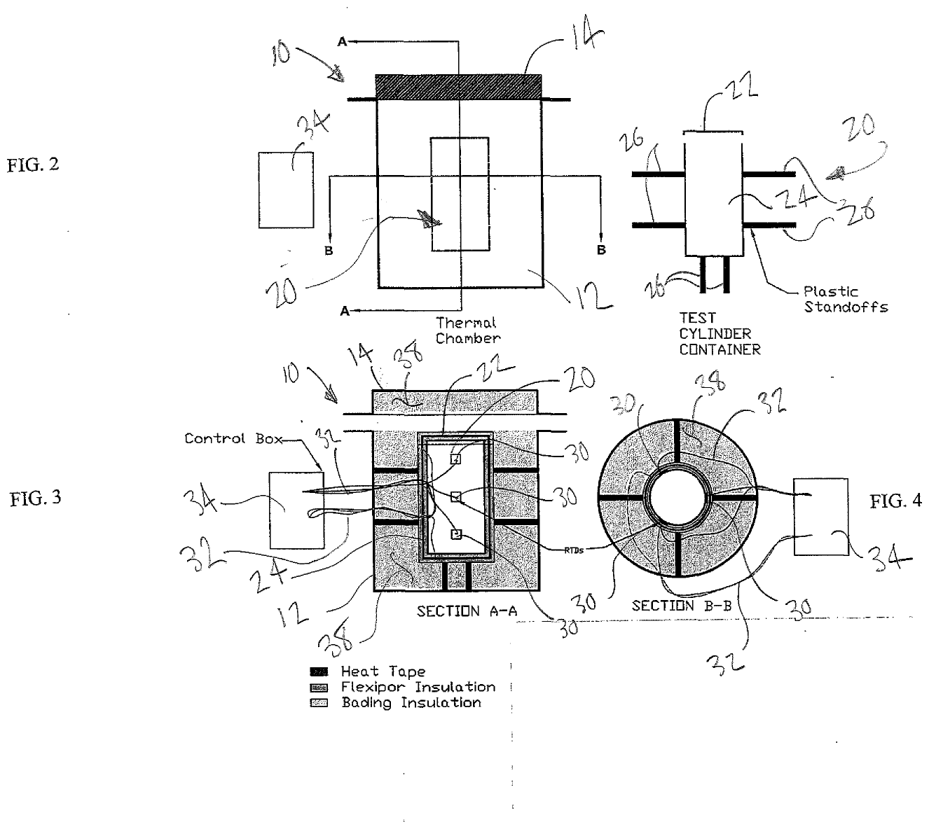

Referring now to FIGS. 1 and 2, an overall view of the thermal chamber 10 and test cylinder container 20 is shown. The thermal chamber 10 includes a body 12 into which the test cylinder container 20 is placed and which is sealed into the body 12 by a cover 14.

The test cylinder container 20 includes a body 22 and cover 24. The test cylinder container 20 is sized to receive a test cylinder of concrete to be tested. Standoffs 26, preferably plastic or some other suitable low heat conducting material, extend outwardly from the side and bottom of the test cylinder container 20 and position the test cylinder container 20 in a centered position in the thermal chamber 10. Note also the spacing of the test cylinder container 20 from the bottom surface of the thermal chamber 10.

As shown in FIGS. 3 and 4, the test cylinder container 20 includes a means of determining the temperature of the concrete test cylinder. One suitable temperature sensing means is resistance temperature detectors 30 (RTDs) positioned in spaced-apart locations on the test cylinder container 20 with leads 32 to a control box 34 containing a data storage device, a suitable computing device and read outs. The RTD's or other temperature sensors 30 may be battery-powered or connected to an electrical service. The control box 34 may include wireless capability for transmitting data to a remote location, for example a laptop, smart phone or tablet.

The body 12 and cover 14 of the thermal chamber 10 are heavily insulated with batting insulation 38 to reduce thermal loss through the walls of the thermal chamber 10. The test cylinder container 20 is also insulated to reduce thermal loss through the walls of the test cylinder container 20. One suitable insulation material is WDS® Flexipor®, manufactured by Morgan Advanced Materials, a microporous insulation material with an extremely low coefficient of thermal conductivity. WDS® Flexipor® consists of inorganic silicates, such as fumed silica, opacifiers for minimizing infrared radiation and reinforcing glass fibers. WDS® Flexipor® is produced with temperature resistant soluble fi62ber paper on both sides and wrapped in a PE film, for purposes of flexibility.

By way of example only, the test cylinder container 20 is constructed of thin stainless steel sheeting, has an interior and exterior diameter of approximately 7 inches (18 cm) and a height of 12 inches (30.5 cm). The thermal chamber 10 has an exterior and interior diameter of approximately 13 inches (33 cm) and a height of 18 inches (46 cm), which includes the cover 14, which has a height of 3 inches (7.6 cm). The nominal diameter of a concrete test cylinder is 6 inches (15 cm) in diameter by 12 inches (30.5 cm) in height. It is possible to use a test colander that is 4 inches (10 cm) in diameter and 8 inches (20 cm) in height.

An adiabatic concrete calorimeter is described above. Various details of the invention may be changed without departing from its scope. Furthermore, the foregoing description of the preferred embodiment of the invention and the best mode for practicing the invention are provided for the purpose of illustration only and not for the purpose of limitation--the invention being defined by the claims.

I claim:

1. An adiabatic concrete calorimeter, comprising:
a. a thermal chamber, including a cover;
b. an insulated test cylinder container for containing a cylindrical concrete sample to be tested by being inserted and sealed into the thermal chamber; and
c. a plurality of temperature sensors positioned in spaced-apart locations on the test cylinder container, and leads connecting the sensors to a control box containing a data storage device.

2. An adiabatic concrete calorimeter for measuring quantity of heat and rate of heat generation from a concrete sample, comprising:
a. an test container for containing the concrete sample;
b. a thermal chamber having one open end for receiving the test container;
c. a cover adapted to seal the test container into the thermal chamber; and
d. a plurality of temperature sensors positioned in spaced-apart locations on the test container.

3. The adiabatic concrete calorimeter according to claim 2, wherein the sensors are resistance temperature detectors.

4. The adiabatic concrete calorimeter according to claim 2, wherein the sensors are connected to a control box and the control box is adapted to communicate data generated by the sensors to a device capable of storing the data.

5. The adiabatic concrete calorimeter according to claim 4, wherein leads connect the sensors to the control box.

6. The adiabatic concrete calorimeter according to claim 4, wherein the sensors are adapted to wirelessly transmit the data.

7. The adiabatic concrete calorimeter according to claim 2, wherein the concrete sample and the test container have a same cross-sectional shape.

8. The adiabatic concrete calorimeter according to claim 6, wherein the cross-sectional shape is a circle.

9. The adiabatic concrete calorimeter according to claim 2, wherein the test container has a plurality of standoffs extending outwardly from an outside perimeter of a side of the test container and downwardly from a bottom of the test container, adapted to position the test container inside of the thermal chamber and provide spacing between the thermal chamber and the test container.

10. The adiabatic concrete calorimeter according to claim 8, wherein the standoffs are made of a low heat conducting material.

11. The adiabatic concrete calorimeter according to claim 2, wherein the standoffs position the test container in a center of a cross-sectional shape of the thermal chamber.

12. The adiabatic concrete calorimeter according to claim 2, wherein the thermal chamber and the cover include insulation.

13. The adiabatic concrete calorimeter according to claim 2, wherein the concrete sample, the test container, and the thermal chamber have a circular cross-sectional shape.

14. A method for measuring quantity of heat and rate of heat generation from a concrete sample, the method comprising:
a. providing an adiabatic concrete calorimeter having a test container positioned within an insulated thermal chamber;
b. inserting the concrete sample into the test container;
c. sealing the sample and the test container within the thermal chamber; and
d. generating data from a plurality of sensors positioned in spaced-apart locations on the test container.
